(19) United States
(12) Patent Application Publication
(10) Pub. No.: US 2004/0132044 A1
(43) Pub. Date: Jul. 8, 2004

(54) Magnetic beads and uses thereof

(76) Inventor: Menachem Ritterband, Nes Ziona (IL)

Correspondence Address: Anthony Castorina, G E Erhlich, Suite 207, 2001Jefferson Davis Highway, Arlington, VA, 22202, US

(21) Appl. No.: 10/476,182

(22) PCT Filed: May 5, 2002
(86) PCT No.: PCT/IL02/00350

(51) Int. Cl. C12Q 1/68

(52) U.S. Cl. 435/6

(57) ABSTRACT

A kit for separating a plurality of analytes in admixture, the kit comprising a plurality of beads each of the plurality of beads having a predetermined and different magnetic response to a magnetic field and each of the beads further having a predetermined affinity to one analyte of the plurality of analytes, such that each magnetic response corresponds to one affinity.

### FIELD AND BACKGROUND OF THE INVENTION

The present invention relates to magnetic beads and, more particularly, to magnetic beads having predetermined and different magnetic responses to a magnetic field and further having predetermined affinities to a plurality of analytes. In one embodiment, the beads are differently responsive to a non-uniform magnetic field. The present invention further relates to (i) a kit incorporating the magnetic beads, (ii) an apparatus for separating the beads, (iii) a system incorporating the kit and apparatus and (iv) a variety of uses of the magnetic beads of the invention.

Various procedures are commonly employed to determine the absence, presence and/or concentration of substances of clinical or research significance which may be present in biological fluids such as urine, whole blood, plasma, serum, sweat, saliva and other body fluids or homogenized tissues. Such substances are commonly referred to as analytes, which include specific binding partners, e.g., antibodies or antigens, drugs, hormones, receptors and the like.

An antibody is a molecule produced by the immune system of an animal, typically in response to the presence a foreign entity such as a pathogen. An antibody forms very strong chemical bonds to a particular portion of the foreign entity, known as antigen; a single foreign entity may have several different antigens, where any particular antibody binds to a single antigen. This recognition and subsequent binding are among the initial stages in an immune response.

In some diagnostic procedures, labeled antibodies, specific for an analyte of interest, are applied to a strip of absorbent material through which labeled antibodies in solution can flow via capillarity. By immobilizing a test sample in a particular portion of the strip, i.e. capture zone, and measuring the amount of labeled antibody which is captured thereat through specific binding, the concentration of analyte in the test sample can be semi-quantitatively determined.

Over the past few decades, small particles, also known in the literature as beads, have become a powerful tool for determining the absence, presence and/or concentration of analytes. Beads are used in numerous biochemical studies such as diagnostic, cell-separation, purification and the like. Columns with various beads are used for affinity, size exclusions and ionic strength separation and purification.

For example, beads are useful for isolation of rare cells from a heterogeneous cell population. The cell suspension is mixed with a specific antibody that has been conjugated to small sized beads, which bind to specific markers unique to the rare cell. Subsequently the beads are collected as a homogeneous group by an outer manipulation, e.g. ultra centrifugation, filtration and the like. It will be appreciated in this respect that manipulation by centrifugation and filtration are relatively complicated, time consuming and costly procedure.

In some purification systems, an analyte of interest is purified from a solution including a variety of analytes by flowing through a packed column of beads contained within a glass or metal tube. The beads are prepared with an affinity to the analyte of interest, thereby allowing the solution to flow away leaving behind the analyte of interest which binds to the beads.

In these systems, however, the separation medium is compacted within the packed column, hence the system has limited capacity and the flow rate in such systems is inherently low.

For efficient separation, the beads must be sufficiently small (typically in a sub-micro or even a sub-nano scale) so that the suspension period of the beads would be long. In addition, the smallness of the beads provides a relatively large reactive surface area and increases the collisions rate of the beads with the target analyte in solution.

Magnetic separation of analytes is a method in which magnetic beads or particles are used as a mobile solid phase. In this method, the beads are prepared with certain magnetic properties, such as dia-, para- or even ferro-magnetism. Magnetic beads can be collected and transferred from one medium to another, using an external magnetic field which may be generated by a permanent magnet or an electromagnet. Magnetic beads are typically used for isolation of a wide range of analytes including, proteins, nucleic acids, viruses, bacteria, cells and the like.

In order to enable a magnetic bead to be used for the immobilization or isolation of substrates, a suitable affinity moiety is applied to the bead. The affinity moiety may be adsorbed onto the surface of each bead or it can be bound, e.g., by covalent linking, to a functionalized groups on the bead. Numerous outer coatings for functional groups binding enlarge the utilization and manipulation of magnetic beads.

Magnetic separation of analytes is known to have high sensitivity, high throughput and to result in high purity. In addition, the use of beads which are responsive to an external magnetic field, greatly simplifies the required technical procedures and manipulations, resulting in cost reduction. A typical example of magnetic separation is magnetic cell separation. Compared to other cell separation methods, magnetic cell separation is characterized by increased recovery and viability of the isolated cell populations.

However, the magnetic beads performance lacks a major important feature, the essence of which is that within a multi-analyte specimen, each group of magnetic beads containing a particular affinity moiety must be reacted separately. Thus, in cases in which a few target analytes are to be detected or purified, the analytes specimen is divided into a plurality of different containers, wherein in each container a different magnetic bead is used for the separation process.

A skilled artisan would appreciate that although this technique achieves a simultaneous effect, each container should be treated and handled separately, resulting in waste of time, specimen and reagents.

The present invention provides solutions to the problems associated with prior art techniques aimed at the separation and/or depletion of a plurality of analytes.

### SUMMARY OF THE INVENTION

According to one aspect of the present invention there is provided a kit for separating a plurality of analytes in admixture, the kit comprising a plurality of beads each having a predetermined and different magnetic response to a magnetic field and each of the beads further having a predetermined affinity to one analyte of the plurality of analytes, such that each magnetic response corresponds to one affinity.

According to another aspect of the present invention there is provided a method of separating a plurality of analytes present in an admixture, the method comprising: (a) providing a plurality of beads each having a predetermined and different magnetic response to a magnetic field, and a predetermined affinity to one analyte, wherein each magnetic response corresponds to one affinity; (b) adding the beads to the admixture under conditions for affinity binding of each of the plurality of analytes to a respective bead; and (c) for each magnetic response, applying a magnetic field having a strength in accordance with the magnetic response, so as to provide a motion of at least one of the beads, thereby differentially separating the analytes from the admixture.

According to further features in preferred embodiments of the invention described below, the method further comprising purifying the plurality of analytes from the beads.

According to still further features in the described preferred embodiments the purifying comprises subjecting the beads to a mechanical operation, so as to spread the beads.

According to still further features in the described preferred embodiments the mechanical operation is selected from the group consisting of shaking, agitating and vibrating.

According to still further features in the described preferred embodiments purifying comprises subjecting the beads to a wash buffer.

According to yet another aspect of the present invention there is provided an apparatus for separating a plurality of beads each of the plurality of beads having a predetermined and different magnetic response to a magnetic field, the apparatus comprising a mechanism for generating a magnetic field having a strength in accordance with each of the magnetic responses, so as to provide a motion of at least one of the beads, thereby to differentially separate the plurality of beads.

According to still another aspect of the present invention there is provided a system for separating a plurality of analytes present in an admixture, the system comprising: (a) a plurality of beads each of the plurality of beads having a predetermined and different magnetic response to a magnetic field, and a predetermined affinity to one analyte of the plurality of analytes, wherein each magnetic response corresponds to one affinity; (b) a container for holding the admixture and the beads under conditions for affinity binding of each of the plurality of analytes to a respective bead; and (c) a mechanism for generating a magnetic field having a strength in accordance with each of the magnetic responses, so as to provide a motion of at least one of the beads, therebv to differentially separate the plurality of beads.

According to further features in preferred embodiments of the invention described below, the analytes are dissolved, suspended or emulsed in a solution.

According to still further features in the described preferred embodiments the analytes are selected from the group consisting of proteins, nucleic acids, viruses, bacteria and cells.

According to still further features in the described preferred embodiments the beads are selected from the group consisting of porous beads and nonporous beads.

According to still further features in the described preferred embodiments the beads are substantially spherical.

According to still further features in the described preferred embodiments the beads are made of a combination of different paramagnetic materials.

According to still further features in the described preferred embodiments the beads are made of a combination of a paramagnetic material and a non-paramagnetic material.

According to still further features in the described preferred embodiments the combination is selected so as to obtain the predetermined and different magnetic response.

According to still further features in the described preferred embodiments a diameter of the beads is selected so as to optimize a resolution of the magnetic response.

According to still further features in the described preferred embodiments the diameter is in a nanometer scale.

According to still further features in the described preferred embodiments the beads are formed by compaction.

According to still further features in the described preferred embodiments each of the beads includes an affinity moiety.

According to still further features in the described preferred embodiments the affinity moiety is capable of binding to an analyte by means of an ionic linkage or a non-ionic linkage.

According to still further features in the described preferred embodiments the affinity moiety is capable of binding to an analyte by means of a covalent linkage or a non-covalent linkage.

According to still further features in the described preferred embodiments the affinity moiety is adsorbed onto a surface of the beads.

According to still further features in the described preferred embodiments the affinity moiety is covalently linked to the beads.

According to still further features in the described preferred embodiments the affinity moiety is selected from the group consisting of a nucleic acid, an antibody, an antigen, a receptor, a ligand, an enzyme, a substrate and an inhibitor.

According to still further features in the described preferred embodiments the mechanism for generating a magnetic field sequentially increases the magnetic field strength.

According to still further features in the described preferred embodiments the mechanism for generating a magnetic field comprises a plurality of permanent magnets.

According to still further features in the described preferred embodiments the mechanism for generating a magnetic field comprises at least one electromagnet.

According to still further features in the described preferred embodiments the mechanism for generating a magnetic field comprises an electromagnetic stick.

According to still further features in the described preferred embodiments the magnetic field is substantially localized within a domain uniquely selected for each magnetic response.

According to still further features in the described preferred embodiments the magnetic field is characterized by a gradient with respect to a predetermined axis.

According to still further features in the described preferred embodiments the system further comprising a purification mechanism for purifying the plurality of analytes.

According to still further features in the described preferred embodiments the purification mechanism is a wash buffer.

According to still further features in the described preferred embodiments the beads have predetermined surface characteristics favoring a wash buffer, hence the beads are characterized by an enhanced contact with the wash buffer.

According to still further features in the described preferred embodiments the wash buffer is selected from the group consisting of an acid, a base, a salt, a denaturant, an oxidant and a reducing agent.

The present invention successfully addresses the shortcomings of the presently known configurations by providing a system, kit, apparatus and method for separating a plurality of analytes through a single and simple procedure. The system enjoys properties far exceeding those characterizing prior art systems.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a kit for separating a plurality of analytes, comprising a plurality of beads, according to the present invention;

FIG. 2 is a an apparatus for separating the plurality of beads, according to the present invention;

FIG. 3 is a system for separating a plurality of analytes, according to the present invention;

FIG. 4 is an illustration of a strength of an external magnetic field having a gradient, according to the present invention;

FIG. 5 is an illustration of a spatial separation of beads, using a magnetic field gradient, according to the present invention; and

FIG. 6 is an illustration of a spatial separation of beads, using an electromagnetic stick, according to the present invention.

### DESCRIPTION OF THE PREFERRED EMBODIMENTS

The present invention is of magnetic beads having predetermined and different magnetic responses to a magnetic field and further having predetermined affinities to a plurality of analytes which can be used for simultaneous biochemical studies and diagnostics. Specifically, the present invention can be used to simultaneously isolate a wide range of analytes including, but not limited to, proteins, nucleic acids, bacteria, viruses, cells and the like. The present invention is further of (i) a kit incorporating the magnetic beads, (ii) an apparatus for separating the beads, (iii) a system incorporating the kit and apparatus and (iv) a variety of uses of the magnetic beads of the invention.

The principles and operation of magnetic beads and uses thereof according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

All materials in nature posses some kind of magnetic properties which are manifested by a force acting on a specific material when present in a magnetic field. These magnetic properties, which originate from the sub-atomic structure of the material, are different from one substrate to another. The direction as well as the magnitude of the magnetic force is different for different materials. Whereas the direction of the force depends only on the internal structure of the material, the magnitude depends both on the internal structure as well as on the size (mass) of the material.

The internal structure of the materials in nature, to which the magnetic characteristics of matter are related, is classified according to one of three major groups: diamagnetic, paramagnetic and ferromagnetic materials, where the strongest magnetic force acts on ferromagnetic materials. In terms of direction, the magnetic force acting on a diamagnetic material is in opposite direction than that of the magnetic force acting on a paramagnetic or a ferromagnetic material.

When placed in external magnetic field, a specific material acquires a non-zero magnetic moment per unit volume, also known as a magnetization, which is proportional to the magnetic field vector. For a sufficiently strong external magnetic field, a ferromagnetic material, due to intrinsic non-local ordering of the spins in the material, may retain its magnetization, hence to become a permanent magnet. As opposed to ferromagnetic materials, both diamagnetic and paramagnetic materials loose the magnetization once the external magnetic field is switched off.

While conceiving the present invention, it has been realized that the above phenomena may be exploited for the purpose of analytes separation. More specifically, because different substrates response differently to an external magnetic field, it is possible to create various alloys, which have almost the same density, but possess essential differences in their response to an external magnetic field. Hence, magnetic beads having different magnetic characteristics may be used as a kit for a differential separation of a plurality of analytes.

Thus, according to the present invention there is provided a kit, generally referred to herein as kit 10, for separating a plurality of analytes in admixture. The analytes to be separated by kit 10 may be any plurality of analytes, such as, but not limited to, proteins, nucleic acids, viruses, bacteria and cells.

Kit 10 includes a plurality of beads each having a predetermined and different magnetic response to a magnetic field.

As used herein, the phrase “a plurality of beads” refers to a plurality of homogenous bead populations.

The different magnetic responses allow differential separation of the beads as further detailed hereinunder. Each magnetic response can be uniquely quantified by a magnetic field threshold, below which the magnetic force acting on the corresponding bead is negligible. In addition to the different magnetic responses, each of the beads further having a predetermined affinity to one of the analytes, so as to uniquely pair a unique magnetic response with a unique affinity to an analyte for each bead population, hence a separation of the beads in accordance with a magnetic response, is equivalent to a separation of the beads in accordance with the corresponding affinities.

According to a preferred embodiment of the present invention each of the beads includes an affinity moiety, which is capable of binding to an analyte. The affinity moiety may be, for example, a nucleic acid, an antibody, an antigen, a receptor, a ligand, an enzyme, a substrate and/or an inhibitor.

According to the present invention, the binding of the affinity moiety to the analyte is not limited to any specific type of binding. Thus, the binding can be by means of an ionic linkage or a non-ionic linkage, or by means of covalent linkage or a non-covalent linkage. The affinity moiety can be adsorbed onto a surface of the beads or, alternatively, it can be covalently linked to the beads. In any of the above embodiments, the resulting beads are capable of binding to the analytes in the admixture, hence to serve as a mobile solid phase.

The different responses of the beads to a magnetic field can be obtained, according to the present invention, in more than one way, for example, the beads can be made of a combination of different materials each having different magnetic characteristics, and thereby different responses to the magnetic field.

Alternatively, the different responses to a magnetic field may be achieved by varying a ratio between a magnetic portion and non-magnetic portion within the beads. The ratio may be predetermined by diluting a certain amount of magnetic particles by an appropriate amount of particles which are substantially non-magnetic, thereby providing a compounded material gradient. The non-magnetic portion of the beads may be a substantially magnetically passive metal or a synthetic polymer.

As used herein the phrase magnetic particle refers to a particle which interacts with an external magnetic field, which interaction is realized by force acting on the particle when present in the magnetic field. The phrase non-magnetic particle, as used herein, refers to a particle having negligible or no capability to interact with an external magnetic field.

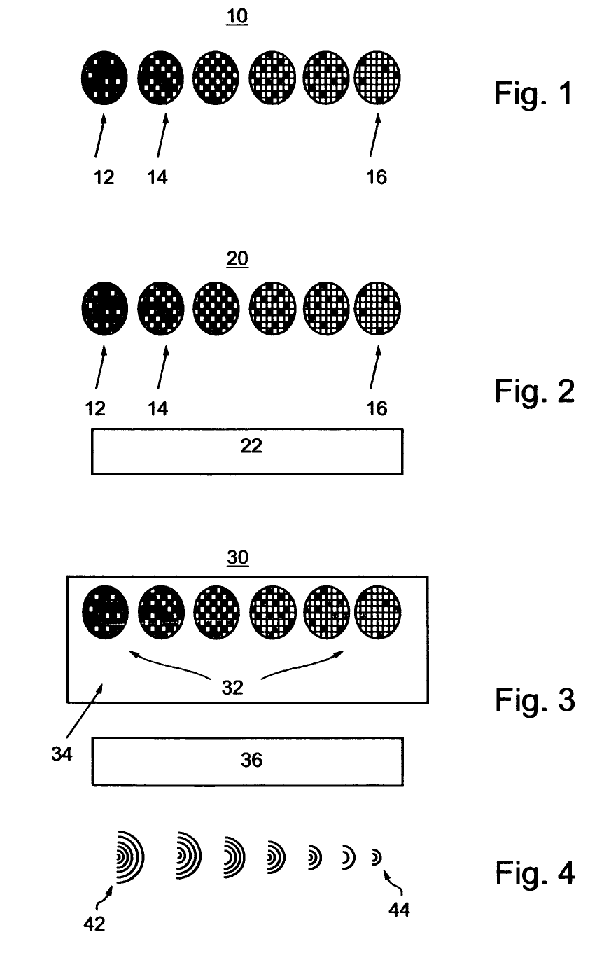

Referring now to the drawings, FIG. 1 illustrates kit 10 in accordance with a preferred embodiment of the present invention. For the purpose of illustration, the beads are represented in FIG. 1 as circles, and the magnetic particles within each bead are represented by dark spots. Hence, in FIG. 1, bead 12 is the most magnetic bead and has high sensitivity to the existence of a magnetic field, bead 14 is less magnetic, and bead 16 has the lowest magnetic response to the existence of a magnetic field.

In order to increase the reactive surface area, the beads are preferably spherical, having a preferred diameter of a nanometer scale, e.g., 1-10 nm. According to preferred embodiments of the present invention the beads may be either porous or nonporous.

The density of the beads should be chosen so as to keep the beads on a suspension form for a sufficiently long time period, e.g., minutes to hours, without rapid gravity sedimentation. The suspension form allows better collisions between the beads and the analytes hence augments the binding efficiency of the beads to their respective analytes via the respective affinity moieties. In addition, the suspension form increases a diffusion rate, hence allows better washing during a certain wash step which may be subsequently executed. On the other hand, in order to prevent beads of being floating, the beads density should be equal or higher than a specific solution in which a specific biochemical reaction is taking place. Thus, the density of the beads is preferably above 1.05 gr/cm³, so as to prevent floating, and preferably below 1.2 gr/cm³, so as to prevent sedimentation. It is to be understood, however that the beads may have higher densities, e.g., to fulfill some industrial demands.

It has been realized by the inventors of the present invention that there is a direct correlation between the diameter of the beads and the number of analytes which may be efficiently separated by kit 10. Hence, the resolution of kit 10 can be controlled by the diameter chosen for the beads. Specifically, larger diameter of the beads corresponds to a larger number of analytes which can be separated.

According to a preferred embodiment of the present invention the beads may have any of the above magnetic characteristics, i.e. dia-para- or ferro-magnetism. Since diamagnetism is a universal characteristic in nature, it is preferable that the beads would be made from paramagnetic or ferromagnetic particles. It should be appreciated, however, that a paramagnetic bead has the advantage that it does not retain its magnetization. Thus, once the external magnetic field is switched off, there are no magnetic forces remaining between the beads. This is particularly important when the analytes need to be released from the beads.

The external magnetic force which acts on the beads aggregates the beads to a relatively compact configuration, and forms some kind of a “bead pile”. As stated, once the magnetic force is terminated, there are no more forces that may leave the beads on an aggregated form. However, although the magnetic force is absent, the beads remains, at least temporarily, in a compact configuration due to certain non repulsion forces on the beads, such as pressure on the bead pile, caused by the solution, directed to ensure the compact form.

The ability of the beads to maintain compact form in the absent of external magnetic force, is referred to herein as Beadophilic.

It is to be understood that a compact configuration is an unstable state of equilibrium. Hence, a small mechanical change in the system breaks the compactness, causes the bead pile to spread, and allows a better liquid flow in between. In other words, the beads are being washed by the liquid. Mechanical changes which may break the compactness are, shaking, agitating, vibrating and the like. It has been found by the inventor that compactness breaking increases the washing efficiency up to a logarithm of 3.

Thus, the unstable state of equilibrium facilitates the release of the analytes from the beads, so as to enable reuse of the beads. It is to be understood that in the embodiments in which the affinity moiety is covalently linked to the beads, the wash step does not remove the affinity moiety and the beads may be used again for a similar process of analytes separation.

The separation of the beads from the analytes may be further improved by providing beads having predetermined surface characteristics which favor the solution in which the wash step is executed. For example, beads having a hydrophilic surface favor binding or releasing in an aqueous solution. Hence, the beads may be prepared so as to enhance a contact between the beads and a conventional wash buffer being used in the wash step.

The physical properties of kit 10 make it an optimal infrastructure component for implementing a method of differential separation of analytes.

Hence, according to another aspect of the invention there is provided a method of separating a plurality of analytes present in an admixture. The method comprising the following steps, which may be executed by an appropriate apparatus or system as is further described hereinunder. A first step includes providing a plurality of beads each having a predetermined and different magnetic response to a magnetic field, and a predetermined affinity to one analyte. The beads may have any combination of the various characteristics of kit 10, as detailed hereinabove. According to a preferred embodiment of the present invention, in a second step the beads are added to the admixture under conditions for affinity binding of each analyte to a respective bead. Finally, in a third step of the method a magnetic field is sequentially applied for each of the magnetic responses.

According to a preferred embodiment of the present invention, the strength of the applied magnetic field is in accordance with the magnetic responses. For example, denoting a bead which responses to an external magnetic field above threshold Bᵢ by bᵢ (i=1, 2 . . . ). Then, assuming that B₁<B₂, in the presence of an external magnetic field having strength B which is larger than B₁ but smaller than B₂, only b₁ “feels” the magnetic field, while b₂ remains insensitive to the existence of the magnetic field. Hence, the magnetic field induces motion on at least one of the beads, thereby allowing to differentially separate each of the analytes from the admixture.

In order to provide an efficient separation of each of the analytes from the admixture, the external field should be applied in a manner that it would attract (or repulse) only one of the beads (and the analyte bound thereto). Thus, according to a preferred embodiment of the present invention the external magnetic field is gradually increased, influencing first the bead characterized by the lowest threshold, then the bead characterized by the next threshold and so on. One ordinarily skilled in the art would appreciate that the bead which is characterized by the smallest threshold has the largest response to a magnetic field.

Referring again to FIG. 1, by applying the weakest magnetic field, bead 12 would be the first to be separated from the admixture, with the first increment of the magnetic field bead 14 will be separated, and the strongest magnetic field will separate bead 16.

It is to be understood that although the embodiments described above are in context of analytes separation, the invention is also capable of purification of a solution containing a plurality of contaminators, or, alternatively, purification of a plurality of analytes present in a contaminated solution. In the former case, the beads are used to remove the contaminators from the solution, while in the latter case the beads isolate the desired analytes, leaving behind the contaminated solution.

Those skilled in the art would appreciate that when analytes are isolated from a contaminated solution, the purification protocol is not completed until the separated analytes are released from the beads.

Hence, according to a preferred embodiment of the present invention, the method further includes a purification step of the analytes from the beads. This step can be done, e.g., by subjecting the beads to a wash buffer. The wash buffer may be, for example, an acid, a base, a salt, a denaturant, an oxidant and/or a reducing agent.

The method described herein can be executed by an appropriate apparatus. Thus, according to an additional aspect of the present invention there is provided an apparatus for separating a plurality of beads, having a predetermined and different magnetic response to a magnetic field.

Reference is now made to FIG. 2 which illustrates the apparatus, generally referred to herein as apparatus 20. Apparatus 20 includes a mechanism 22 for generating a magnetic field. As explained hereinabove, the strength of the magnetic field is in accordance with the magnetic responses of the beads, so as to provide a motion of at least one of the beads, thereby to differentially separate the plurality of beads.

According to a preferred embodiment of the present invention mechanism 22 may be any mechanism for generating a magnetic field, such as, but not limited to, one or more permanent magnets or electromagnets. Alternatively mechanism 22 may be a simple electromagnetic stick for collecting the beads.

In principle, apparatus 20 may be used to separate any plurality of beads having different magnetic responses, for example the beads provided by kit 10. Hence, in accordance with a preferred embodiment of the invention, the beads may further have a predetermined affinity to one analyte of a plurality of analytes, such that, as is further entailed hereinabove, each magnetic response corresponds to one affinity.

The present invention successfully provides a separating or a purifying system, referred to herein as system 30.

Reference is now made to FIG. 3, illustrating system 30, according to the presently preferred embodiment of the invention. System 30 includes a plurality of beads 32 similar to the beads provided by kit 10. Beads 32 serve as a mobile solid phase as already explained above. System 30 further includes a container 34 for holding beads 32 and an admixture of the analytes under conditions for affinity binding of each of the analytes to a respective bead. According to a preferred embodiment of the present invention the analytes may be dissolved, suspended or emulsed in a solution, hence container 32 holds the solution into which beads 32 are added.

System 30 further includes a mechanism 36 for generating a magnetic field. The principles and operation of mechanism 36 are similar to those of mechanism 22 described above with respect to apparatus 20. Hence mechanism 36 serves for providing a motion of at least one of the beads, thereby to differentially separate the plurality of beads, and as a consequence to separate the analytes from the admixture.

The separation procedure, as executed by mechanism 36, is done in a similar way as described above, e.g., by applying a magnetic field, the strength of which gradually increases so as to first separate the most magnetic bead (e.g., bead 12 shown FIG. 1). A skilled artisan will appreciate that a spatial separation of the beads, once magnetized by the relevant magnetic field, can be achieved in more than one way.

A first alternative is illustrated in FIG. 4. According to a preferred embodiment of the present invention, mechanism 36 provides an external magnetic field which is substantially localized within a predetermined domain, which is uniquely selected for each bead. The strength of the magnetic field is represented in FIG. 4 as a bundle of concentric semicircles, where the largest bundle 42 corresponds to the strongest magnetic field, and smallest bundle 44 corresponds to the weakest magnetic field. Hence, the beads are directed by a magnetic field gradient.

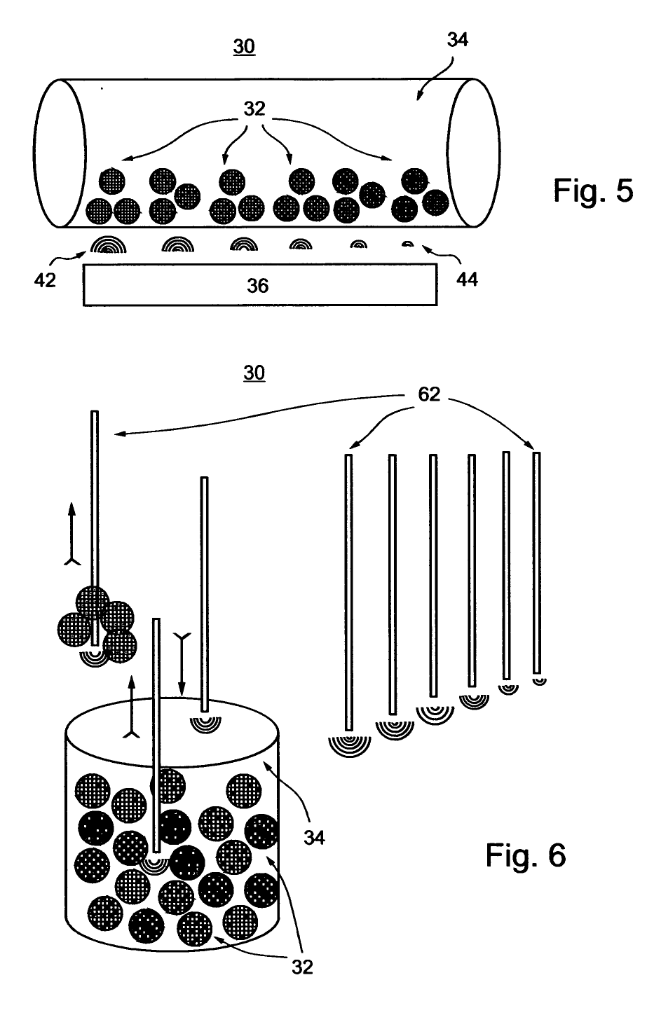

Reference is now made to FIG. 5, illustrating the spatial separation of beads 32, according to the presently preferred embodiment of the invention. Thus, after the gradual increment of the magnetic field as described above, and the formation of a spatial gradient with respect to a longitudinal axis of container 34, beads 32 are spatially separated where the most magnetic bead is entrapped within the domain of the weakest magnetic field, and so forth.

Reference is now made to FIG. 6, illustrating a second alternative for spatial separation of beads 32. As shown in FIG. 6, mechanism 36 can be embodied as an electromagnetic stick 62, used for actual “fishing” of beads 32 from container 34. The use of electromagnetic stick 62 allows controlling the magnetic field strength. Thus, electromagnetic stick 62 is first inserted into container 34 while generating the weakest magnetic field for capturing the most magnetic bead. Once the bead is captured, it can be released to a different tube by switching of the magnetic field. The procedure is iteratively repeated, each time with stronger magnetic field, until all of the beads are displaced from container 34.

Thus, the magnetic beads and the sequential application of magnetic field, as provided by the present invention and described hereinabove, enable both manual and automatic differentiation and separation of analytes based on their different affinity binding.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A kit for separating a plurality of analytes in admixture, the kit comprising a plurality of beads each of said plurality of beads having a predetermined and different magnetic response to a magnetic field and each of said beads further having a predetermined affinity to one analyte of the plurality of analytes, such that each said magnetic response corresponds to one said affinity.

2. The kit of claim 1, wherein the analytes are dissolved, suspended or emulsed in a solution.

3. The kit of claim 1, wherein the analytes are selected from the group consisting of proteins, nucleic acids, viruses, bacteria and cells.

4. The kit of claim 1, wherein said beads are selected from the group consisting of porous beads and nonporous beads.

5. The kit of claim 1, wherein said beads are substantially spherical.

6. The kit of claim 1, wherein said beads are made of a combination of different paramagnetic materials.

7. The kit of claim 6, wherein said combination is selected so as to obtain said predetermined and different magnetic response.

8. The kit of claim 1, wherein the beads are made of a combination of a paramagnetic material and a non-paramagnetic material.

9. The kit of claim 8, wherein said combination is selected so as to obtain said predetermined and different magnetic response.

10. The kit of claim 1, wherein a diameter of said beads is selected so as to optimize a resolution of said magnetic response.

11. The kit of claim 10, wherein said diameter is in a nanometer scale.

12. The kit of claim 1, wherein said beads have predetermined surface characteristics favoring a wash buffer, hence said beads are characterized by an enhanced contact with said wash buffer.

13. The kit of claim 12, wherein said wash buffer is selected from the group consisting of an acid, a base, a salt, a denaturant, an oxidant and a reducing agent.

14. The kit of claim 1, wherein said beads are formed by compaction.

15. The kit of claim 1, wherein each of said beads include an affinity moiety.

16. The kit of claim 15, wherein said affinity moiety is capable of binding to an analyte by means of an ionic linkage or a non-ionic linkage.

17. The kit of claim 15, wherein said affinity moiety is capable of binding to an analyte by means of covalent linkage or a non-covalent linkage.

18. The kit of claim 15, wherein said affinity moiety is adsorbed onto a surface of said beads.

19. The kit of claim 18, wherein said affinity moiety is covalently linked to said beads.

20. The kit of claim 15, wherein said affinity moiety is selected from the group consisting of a nucleic acid, an antibody, an antigen, a receptor, a ligand, an enzyme, a substrate and an inhibitor.

21. A method of separating a plurality of analytes present in an admixture, the method comprising:
(a) providing a plurality of beads each of said plurality of beads having a predetermined and different magnetic response to a magnetic field, and a predetermined affinity to one analyte of the plurality of analytes, wherein each said magnetic response corresponds to one said affinity;
(b) adding said beads to the admixture under conditions for affinity binding of each of said plurality of analytes to a respective bead; and
(c) for each said magnetic response, applying a magnetic field having a strength in accordance with said magnetic response, so as to provide a motion of at least one of said beads, thereby differentially separating the analytes from the admixture.

22. The method of claim 21, wherein the analytes are dissolved, suspended or emulsed in a solution.

23. The method of claim 21, wherein the analytes are selected from the group consisting of proteins, nucleic acids, viruses, bacteria and cells.

24. The method of claim 21, wherein said beads are selected from the group consisting of porous beads and nonporous beads.

25. The method of claim 21, wherein said beads are substantially spherical.

26. The method of claim 21, wherein said beads are made of a combination of different paramagnetic materials.

27. The method of claim 26, wherein said combination is selected so as to obtain said predetermined and different magnetic response.

28. The method of claim 21, wherein the beads are made of a combination of a paramagnetic material and a non-paramagnetic material.

29. The method of claim 28, wherein said combination is selected so as to obtain said predetermined and different magnetic response.

30. The method of claim 21, wherein a diameter of said beads is selected so as to optimize a resolution of said magnetic response.

31. The method of claim 30, wherein said diameter is in a nanometer scale.

32. The method of claim 21, wherein said beads are formed by compaction.

33. The method of claim 21, wherein each of said beads include an affinity moiety.

34. The method of claim 33, wherein said affinity) moiety is capable of binding to an analyte by means of an ionic linkage or a non-ionic linkage.

35. The method of claim 33, wherein said affinity moiety is capable of binding to an analyte by means of covalent linkage or a non-covalent linkage.

36. The method of claim 33, wherein said affinity moiety is adsorbed onto a surface of said beads.

37. The method of claim 36, wherein said affinity moiety is covalently linked to said beads.

38. The method of claim 33, wherein said affinity moiety is selected from the group consisting of a nucleic acid, an antibody, an antigen, a receptor, a ligand, an enzyme, a substrate and an inhibitor.

39. The method of claim 21, wherein said applying said magnetic field is effected by a procedure of sequentially increasing said magnetic field strength.

40. The method of claim 21, wherein said applying said magnetic field is by a plurality of permanent magnets.

41. The method of claim 21, wherein said applying said magnetic field is by at least one electromagnet.

42. The method of claim 21, wherein said applying said magnetic field is by electromagnetic stick.

43. The method of claim 21, w herein said applying said magnetic field is in manner such that said magnetic field is substantially localized within a domain uniquely selected for each said magnetic response.

44. The method of claim 21, wherein said magnetic field is characterized by a gradient with respect to a predetermined axis.

45. The method of claim 21, further comprising purifying the plurality of analytes from said beads.

46. The method of claim 45, wherein said purifying comprises subjecting the beads to a mechanical operation, so as to spread the beads.

47. The method of claim 45, wherein said mechanical operation is selected from the group consisting of shaking, agitating and vibrating.

48. The method of claim 46, wherein said purifying further comprises subjecting the beads to a wash buffer.

49. The method of claim 48, wherein said beads have predetermined surface characteristics favoring a wash buffer, hence said beads are characterized by an enhanced contact with said wash buffer.

50. The method of claim 48, wherein said wash buffer is selected from the group consisting of an acid, a base, a salt, a denaturant, an oxidant and a reducing agent.

51. An apparatus for separating a plurality of beads each of the plurality of beads having a predetermined and different magnetic response to a magnetic field, the apparatus comprising a mechanism for generating a magnetic field having a strength in accordance with each of said magnetic responses, so as to provide a motion of at least one of said beads, thereby to differentially separate the plurality of beads.

52. The apparatus of claim 51, wherein the beads are selected from the group consisting of porous beads and nonporous beads.

53. The apparatus of claim 51, wherein the beads are substantially spherical.

54. The apparatus of claim 51, wherein the beads are made of a combination of different paramagnetic materials.

55. The apparatus of claim 54, wherein said combination is selected so as to obtain the predetermined and different magnetic response.

56. The apparatus of claim 51, wherein the beads are made of a combination of a paramagnetic material and a non-paramagnetic material.

57. The apparatus of claim 56, wherein said combination is selected so as to obtain the predetermined and different magnetic response.

58. The apparatus of claim 51, wherein a diameter of the beads is selected so as to optimize a resolution of said magnetic response.

59. The apparatus of claim 58, wherein said diameter is in a nanometer scale.

60. The apparatus of claim 51, wherein the beads have predetermined surface characteristics favoring a wash buffer, hence said beads are characterized by an enhanced contact with said wash buffer.

61. The apparatus of claim 60, wherein said wash buffer is selected from the group consisting of an acid, a base, a salt, a denaturant, an oxidant and a reducing agent.

62. The apparatus of claim 51, wherein the beads are formed by compaction.

63. The apparatus of claim 51, wherein each of the beads further having a predetermined affinity to one analyte of a plurality of analytes, such that each magnetic response corresponds to one said affinity.

64. The apparatus of claim 63, wherein said analytes are dissolved, suspended or emulsed in a solution.

65. The apparatus of claim 63, wherein said analytes are selected from the group consisting of proteins, nucleic acids, viruses, bacteria and cells.

66. The apparatus of claim 51, wherein each of the beads include an affinity moiety.

67. The apparatus of claim 66, wherein said affinity moiety is capable of binding to an analyte by means of an ionic linkage or a non-ionic linkage.

68. The apparatus of claim 63, wherein said analyte is selected from the group consisting of a protein, a nucleic acid, a virus, a bacterium and a cell.

69. The apparatus of claim 66, wherein said affinity moiety is capable of binding to an analyte by means of covalent linkage or a non-covalent linkage.

70. The apparatus of claim 66, wherein said affinity moiety is adsorbed onto a surface of the beads.

71. The apparatus of claim 70, wherein said affinity moiety is covalently linked to said beads.

72. The apparatus of claim 66, wherein said affinity moiety is selected from the group consisting of a nucleic acid, an antibody, an antigen, a receptor, a ligand, an enzyme, a substrate and an inhibitor.

73. The apparatus of claim 51, wherein said mechanism for generating a magnetic field sequentially increases said magnetic field strength.

74. The apparatus of claim 51, wherein said mechanism for generating a magnetic field comprises a plurality of permanent magnets.

75. The apparatus of claim 51, wherein said mechanism for generating a magnetic field comprises at least one electromagnet.

76. The apparatus of claim 51, wherein said mechanism for generating a magnetic field comprises an electromagnetic stick.

77. The apparatus of claim 51, wherein said magnetic field is substantially localized within a domain uniquely selected for each magnetic response.

78. The apparatus of claim 51, wherein said magnetic field is characterized by a gradient with respect to a predetermined axis.

79. A system for separating a plurality of analytes present in an admixture, the system comprising:
(a) a plurality of beads each of said plurality of beads having a predetermined and different magnetic response to a magnetic field, and a predetermined affinity to one analyte of the plurality of analytes, wherein each said magnetic response corresponds to one said affinity;
(b) a container for holding the admixture and said beads under conditions for affinity binding of each of said plurality of analytes to a respective bead; and
(c) a mechanism for generating a magnetic field having a strength in accordance with each of said magnetic responses, so as to provide a motion of at least one of said beads, thereby to differentially separate the plurality of beads.

80. The system of claim 79, wherein the analytes are dissolved, suspended or emulsed in a solution.

81. The system of claim 79, wherein the analytes are selected from the group consisting of proteins, nucleic acids, viruses, bacteria and cells.

82. The system of claim 79, wherein said beads are selected from the group consisting of porous beads and nonporous beads.

83. The system of claim 79, wherein said beads are substantially spherical.

84. The system of claim 79, wherein said beads are made of a combination of different paramagnetic materials.

85. The system of claim 84, wherein said combination is selected so as to obtain said predetermined and different magnetic response.

86. The system of claim 79, wherein the beads are made of a combination of a paramagnetic material and a non-paramagnetic material.

87. The system of claim 86, wherein said combination is selected so as to obtain said predetermined and different magnetic response.

88. The system of claim 79, wherein a diameter of said beads is selected so as to optimize a resolution of said magnetic response.

89. The system of claim 88, wherein said diameter is in a nanometer scale.

90. The system of claim 79, wherein said beads are formed by compaction.

91. The system of claim 79, wherein each of said beads include an affinity moiety.

92. The system of claim 91, wherein said affinity moiety is capable of binding to an analyte by means of an ionic linkage or a non-ionic linkage.

93. The system of claim 91, wherein said affinity moiety is capable of binding to an analyte by means of covalent linkage or a non-covalent linkage.

94. The system of claim 91, wherein said affinity moiety is adsorbed onto a surface of said beads.

95. The system of claim 94, wherein said affinity moiety is covalently linked to said beads.

96. The system of claim 91, wherein said affinity moiety is selected from the group consisting of a nucleic acid, an antibody, an antigen, a receptor, a ligand, an enzyme, a substrate and an inhibitor.

97. The system of claim 79, wherein said mechanism for generating a magnetic field sequentially increases said magnetic field strength.

98. The system of claim 79, wherein said mechanism for generating a magnetic field comprises a plurality of permanent magnets.

99. The system of claim 79, wherein said mechanism for generating a magnetic field comprises at least one electromagnet.

100. The system of claim 79, wherein said mechanism for generating a magnetic field comprises an electromagnetic stick.

101. The system of claim 79, wherein said magnetic field is substantially localized within a domain of said container, said domain is uniquely selected for each said magnetic response.

102. The system of claim 79, wherein said magnetic field is characterized by a gradient with respect to a predetermined axis.

103. The system of claim 79, further comprising a purification mechanism for purifying the plurality of analytes.

104. The method of claim 103, wherein said purification mechanism is a wash buffer.

105. The system of claim 104, wherein said beads have predetermined surface characteristics favoring a wash buffer, hence said beads are characterized by an enhanced contact with said wash buffer.

106. The system of claim 0.104, wherein said wash buffer is selected from the group consisting of an acid, a base, a salt, a denaturant, an oxidant and a reducing agent.
